(19) United States
(12) Patent Application Publication
(10) Pub. No.: US 2021/0155890 A1
(43) Pub. Date: May 27, 2021

(54) INCUBATOR DEVICE, CELL CULTURE ENVIRONMENT CONTROL SYSTEM, AND CELL CULTURE ENVIRONMENT CONTROL METHOD

(71) Applicants: National University Corporation Kumamoto University, Kumamoto (JP); Ushio Denki Kabushiki Kaisha, Tokyo (JP)

(72) Inventors: Yuta NAKASHIMA, Kumamoto-shi (JP); Kinichi MORITA, Tokyo (JP)

(73) Assignees: National University Corporation Kumamoto University, Kumamoto (JP); Ushio Denki Kabushiki Kaisha, Tokyo (JP)

(21) Appl. No.: 17/046,379

(22) PCT Filed: Apr. 8, 2019
(86) PCT No.: PCT/JP2019/015333

§ 371 Date: Oct. 9, 2020

(30) Foreign Application Priority Data
Apr. 13, 2018 (JP) 2018-077917

(51) Int. Cl. C12M 1/00; C12M 1/34; C12M 1/36

(52) U.S. Cl. CPC C12M 41/14; C12M 41/48; C12M 41/26; C12M 31/10

(57) ABSTRACT

A purpose of the present invention is to provide an incubator device and the like capable of performing measurements in a manner such as to minimize changes in the state of a medium. The incubator device is designed to control the cell culture environment and includes an airtight housing, a light source for irradiating a medium containing seeded cells with light, a light measurement unit for measuring the intensity of light from the medium, and a light guide member for guiding the light from the medium to the light measurement unit. The light source unit, the light measurement unit and the light guide member are placed within the housing.

### TECHNICAL FIELD

The present invention relates to an incubator device and the like for cell culture, and more particularly, to the incubator device and the like for cell culture capable of observing a cell culture state.

### BACKGROUND ART

In cell culture, it is necessary to adjust the culture environment for cell proliferation. Specifically, it is necessary to regulate the physicochemical environment, such as humidity, pH, osmotic pressure, oxygen partial pressure and carbon dioxide partial pressure, and regulate the physiological environment, such as hormone and nutrient concentrations. Such a culture environment, except for temperature, is controlled by a medium (culture medium).

That is, the medium provides the necessary nutrients, growth factors and hormone for cell growth, and also controls the pH and osmotic pressure of the culture solution. Thus, the medium is an important regulator in the adjustment of the culture environment.

Most of common mammalian cellular systems (common mammal cell lines) grow well with pH being 7.4. In order to reduce the influence on cultured cells, it is desired that the pH of the medium is kept constant. The pH of the medium depends on the balance between dissolved carbon dioxide (CO₂) and bicarbonate (HCO₃⁻). Therefore, the pH of the medium varies with CO₂ in the atmosphere (outside air). Therefore, the use of exogenous CO₂ is mandatory when cell culture is performed using a medium. Consequently, the inside environment of the incubator device should be maintained at the optimum temperature and humidity for cell culture, and CO₂ concentration should also be maintained at a predetermined value. In other words, if the pH of the medium deviates from a predetermined value, it is necessary to replace the medium.

On the other hand, in general, the culture process of the cell reaches the stationary phase through the induction phase and the logarithmic growth phase, and eventually moves to the death phase. In the logarithmic growth phase, when the adherent culture type cells extend over the entire surface of the medium such that there is no further place for proliferation, or when the number of the float culture-type cells (suspension cultured cells) exceeds the culture volume (capacity) of the medium, the cell proliferation greatly reduces or completely stops. Therefore, passage is necessary in order to maintain further cell proliferation.

In order to determine the timing of the medium replacement or passage, the medium is usually stained (colored) with a dye such as phenol red. The phenol red is an indicator to know the pH of the medium.

If the color of the medium stained with the phenol red becomes reddish purple, the medium is alkaline. The condition for the medium to become alkaline is, for example, when at least some of the cells in the culture are dead, the CO₂ concentration in the incubator device is below a predetermined value, or the circulation of CO₂ in the incubator device is stagnant, resulting in inadequate pH control of the medium.

In such cases, the medium must be replaced with a new medium to reculture the new cells or to verify the CO₂ delivery status in the incubator (i.e., the CO₂ concentration and an operating status of a CO₂ circulating mechanism).

On the other hand, if the color of the medium stained with the phenol red becomes yellow, the medium is acidic. The medium becomes acidic when the number of cells in the logarithmic growth phase increases and the metabolites of the cells (mainly lactic acid) accumulate in the medium. Alternatively, the medium becomes acidic when impurities are present in the medium.

In this case, it is necessary to replace the medium with a new medium or carry out the passage. Especially, if impurities enter the medium in a laboratory carrying out a genetic research, the laboratory is closed for about one month. Then, the laboratory is sterilized by irradiating ultraviolet rays for twenty-four hours continuously.

Conventionally, the color of the medium is visually checked. Therefore, the determination of the timing for the medium replacement and the like is affected by the experience, sensation and the like of an operator, and the consistent determination is not always made.

Due to the above-described circumstances, there has been a demand for a technique for quantitatively and automatically monitoring a state of a medium without depending upon visual observations by the operator. As a method of monitoring a cell culture state by a measuring device, for example, the following is known.

Patent Literature Document 1 discloses a technique for monitoring a culture state, i.e., it discloses a culture monitor in which a part of a culture solution in a cell liquid culture is taken out and a substance produced by a cell contained in the culture solution is measured by a sensor.

### LISTING OF REFERENCES

### Patent Literature Documents

Patent Literature Document 1: Japanese Patent Application Laid-Open Publication No. 2002-148258

Patent Literature Document 2: Japanese Patent No. 5665811

Patent Literature Document 3: Japanese Patent Application No. 2017-131126

### SUMMARY OF THE INVENTION

### Problems to be Solved by the Invention

In the above-described conventional technique, although quantitative monitoring is possible, a portion of the culture solution is taken out. Thus, the state of the medium changes so much to be ignored every time monitoring is performed.

Accordingly, it is an object of the present invention to provide an incubator device and the like which can conduct the measuring without changing the state of a medium as much as possible.

### Solution to the Problems

According to a first aspect of the present invention, there is provided an incubator device that is designed to control a cell culture environment. The incubator device includes a housing having airtightness, a light source unit for irradiating at least one medium, in which cells are seeded (inoculated), with light, a light measuring unit for measuring intensity of the light from the medium(s), and a light guide member for guiding the light from the medium(s) to the light measuring unit. The light source unit, the light measuring unit and the light guide member are disposed inside the housing.

A second aspect of the present invention is directed to the incubator device of the first aspect, and the light guide member includes a light guide path for transmitting light, and a light shielding unit provided around the light guide path and configured to shield light. The light shielding unit includes a silicone resin and light absorbing particles dispersed in the silicone resin.

A third aspect of the present invention is directed to the incubator device of the first or second aspect, and the incubator device further includes a signal transmitting unit for transmitting the intensity of the light measured by the light measuring unit toward an external receiver.

A fourth aspect of the present invention is directed to the incubator device of any one of the first to third aspects, and the light source unit has a plurality of light sources corresponding to a plurality of the above-mentioned mediums arranged in a microplate, respectively.

A fifth aspect of the present invention is directed to the incubator device of any one of the first to fourth aspects, and the light source unit has one or more white LED light sources, and the light measuring unit includes one or more RGB color sensors.

According to a sixth aspect of the present invention, there is provided a cell culture environment control system for controlling a cell culture environment. The system includes: an incubator device according to any one of the first to fifth aspects; an absorbance calculation unit that calculates an absorbance from light intensity measured by the light measuring unit of the incubator device; a pH calculation unit that calculates a pH from the absorbance calculated by the absorbance calculation unit; and a carbon dioxide concentration control unit that maintains a carbon dioxide concentration inside the housing when the pH calculated by the pH calculation unit is within a range from a lower limit value to an upper limit value, increases the carbon dioxide concentration inside the housing when the pH calculated by the pH calculation unit is larger than the upper limit value, and decreases the carbon dioxide concentration inside the housing when the pH calculated by the pH calculation unit is smaller than the lower limit value.

A seventh of the present invention is directed to a cell culture environment control system of the sixth aspect, and the system further includes: a turbidity calculation unit that calculates turbidity from the light intensity measured by the light measurement unit; a medium condition determining unit that determines that disposal of the medium is necessary when the pH calculated by the pH calculation unit is smaller than the lower limit value and the turbidity calculated by the turbidity calculation unit is equal to or larger than a threshold value, determines that replacement or passage of the medium is necessary when the pH calculated by the pH calculation unit is smaller than the lower limit value and the turbidity calculated by the turbidity calculation unit is lower than the threshold value, and determines that replacement of the medium is necessary when the pH calculated by the pH calculation unit is continuously larger than the upper limit value for a predetermined period even if the carbon dioxide concentration inside the housing is increased by the carbon dioxide concentration control unit; and a medium information display unit that displays necessity of the replacement, the passage or the disposal of the medium determined by the medium condition determining unit.

According to an eighth aspect of the present invention, there is provided a cell culture environment control method for controlling a cell culture environment. The method includes the sealing step of loading a medium, in which cells are seeded and which is stained with a reagent, in a sealed space, the light intensity measuring step of irradiating the medium with light to measure intensity of light from the medium while maintaining the sealed space, the pH calculation step of calculating a pH of the medium from the intensity of the light measured in the light intensity measuring step, and the carbon dioxide concentration control step of maintaining a carbon dioxide concentration inside the housing when the pH calculated in the pH calculation step is within a range from a lower limit value to an upper limit value, increasing the concentration of carbon dioxide inside the housing when the pH calculated in the pH calculation step is larger than the upper limit value, and reducing the carbon dioxide concentration inside the housing when the pH calculated in the pH calculation step is smaller than the lower limit value.

### Advantageous Effects of the Invention

According to each aspect of the present invention, it is possible to quantitatively measure the pH or the turbidity which is an indicator of the state (condition) of the medium while allowing the control to the cell culture environment. Thus, it is possible to quantitatively determine the state of the medium and to perform treatment such as passage and medium replacement at an appropriate timing.

Conventionally, confirmation of the color of the medium has been performed by visual observation of an operator or measurement of a collected culture solution. Thus, it is difficult to continuously perform the confirmation of the color of the medium, and the medium color confirmation has been intermittently performed at every constant timing. Therefore, the timing of passage delayed sometimes. According to each aspect of the present invention, it is possible to automatically and continuously monitor the state of the medium.

Conventionally, when it has been found that a medium replacement or the like is unnecessary after the petri dish in which the medium is placed is taken out from the incubator device, the petri dish is again placed inside the incubator device. As the petri dish that has the medium therein is put into and out of the incubator device in this manner, there has been a case where a defect such as contamination of impurities into the medium occurs. According to each aspect of the present invention, it is not necessary to take in and out the medium for confirmation of the culture state, and it is possible to reduce the chance of impurity contamination.

According to the second aspect of the present invention, since the light incident on the light shielding portion is absorbed by the light absorbing particles and hardly returns to the light guide path from the light shielding portion, complex multiple reflections of stray light hardly occur. Thus, it is possible to perform the optical measurement with the ratio of the detected light to noise light, such as unwanted external light and stray light, being sufficiently high. As a result, it is not necessary to shield the entire housing in order to block the external light. Accordingly, it is possible to achieve both suppressing the noise light and visually observing the cells.

According to the third aspect of the present invention, it is possible to obtain the measurement data measured by the light measuring unit in the incubator device, without opening and closing the incubator device. Also, it becomes possible to observe the culture environment of the cells in real time.

According to the fourth aspect of the present invention, since there is one light source corresponding to each medium, there is no need to move the light source(s). This enables highly reproducible measurements. It is also possible to reduce the size of the device, as compared with providing a structure for moving a light source, such as a common microplate reader, or a microplate or the like.

According to the fifth aspect of the present invention, it is possible to simultaneously measure the absorbance and turbidity of the medium. In addition, since the light of the wavelength emitted by the white LED light source is less cytotoxic and the light source itself does not reach a high temperature, it is possible to suppress the influence of the light measurement on the culture environment of the cells. Furthermore, the downsizing of the device becomes possible.

According to the sixth aspect of the present invention, it is possible to provide a cell culture environment control system which can easily control carbon dioxide concentration.

According to the seventh aspect of the present invention, it is possible to provide a cell culture environment control system capable of quantitatively determining the necessity of replacement, passage and disposal of a medium.

### BRIEF DESCRIPTION OF THE DRAWINGS

FIG. 1 is a view showing a structure of an incubator device according to a first embodiment of the present invention.

FIG. 2 is a view showing a structure of an incubator device according to a second embodiment of the present invention.

FIG. 3 is a scatter plot of turbidity versus absorbance of the medium.

FIG. 4 is a set of diagrams showing the absorbance measured in the incubator device of the second embodiment.

FIG. 5 is a set of diagrams showing the absorbance measured by a conventional spectrophotometer.

### DESCRIPTION OF EMBODIMENTS

Referring to the drawings, embodiments of the incubator device of the present invention will now be described.

### First Embodiment

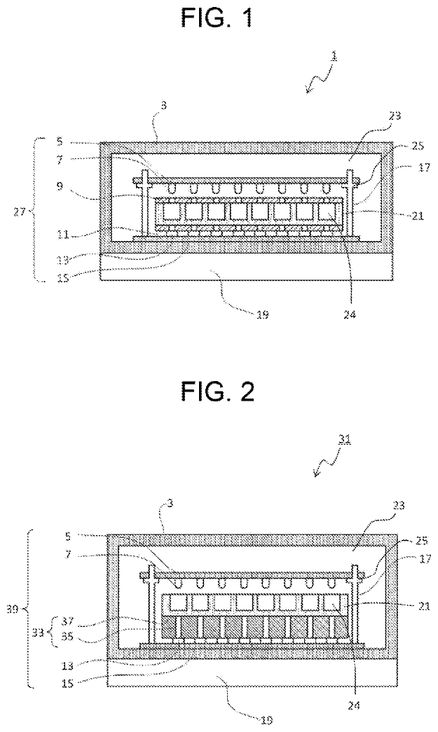

FIG. 1 shows an exemplary configuration of an incubator device 1 according to an embodiment of the present invention (an example of the “incubator device” in the claims). The incubator device 1 includes a housing 3 (an example of the “housing” in the claims), an LED drive board or substrate 5, a plurality of LEDs 7 (an example of the “light source unit” in the claims), a first aperture substrate 9, a second aperture substrate 11, a plurality of sensors 13 (an example of the “light measuring unit” in the claims), a sensor drive board or substrate 15, support members 17, and a power supply-control-and-communication unit 19. The LED drive board 5, the LEDs 7, the first aperture substrate 9, the second aperture substrate 11, the sensors 13, the sensor substrate 15, and the support portions 17 are placed in the housing 3.

The incubator device 1 has a cell culture space 23 in which a medium storage container 21 is disposed, and can control the temperature and humidity of the cell culture space 23 to conditions suitable for cell culture. The incubator device 1 also has a function of controlling the CO₂ concentration inside the cell culture space 23 in order to maintain the pH value of the medium 24 at a value suitable for cell culture. Incidentally, in FIG. 1, a control mechanism for controlling the temperature, the humidity and the CO₂ concentration is not shown.

The temperature/humidity/CO₂ concentration control mechanism is powered and controlled by the power supply-control-and-communication unit 19. In FIG. 1, the power supply-control-and-communication unit 19 is disposed below the housing 3 of the incubator device 1, but the present invention is not limited to such arrangement.

Characteristics of the incubator device 1 are to include a light source for emitting light to the medium storage container 21, which contains the medium 24 with the cells being seeded therein, and the sensors 13 for receiving the light, which is emitted from the light source and passes through the medium 24 and the medium storage container 21, and measuring the light intensity. FIG. 1 shows an example in which a microplate is used as the medium storage container 21.

On the bottom surface of the cell culture space 23 inside the housing 3, the sensors 13 (e.g., photodiodes) are provided such that the sensing surfaces of the sensors face upward. The sensor drive board 15 is connected to the sensors 13 for power supply to the sensors and operation control of the sensors. The sensors 13 on the sensor drive substrate 15 are arranged such that the sensors correspond to the number and locations of the wells of the microplate 21, respectively.

The microplate 21 is disposed above the sensors 13. Further, the LEDs 7 are provided above the microplate 21 such that the LEDs 7 face the microplate 23 and the sensors 13. The LED drive board 5 for power supply and operation control is connected to the LEDs 7.

The LED drive board 5 and the sensor drive board 15 are positioned by the support members 17 such that the positions of the sensors 13 and the positions of the LEDs 7 correspond to each other and such that the upper surface of the microplate 21 and the LEDs 7 are apart from each other with an appropriate distance. In the configuration shown in FIG. 1, each of the support members 17 is a cylindrical structure having a flange portion 25 near the top. The flange portions 25 decide the height (vertical position) of the LED drive board 5 in the housing 3, which is measured from the bottom surface of the cell culture space 23 in the housing 3. As the cylindrical structures penetrate the through holes formed in the LED drive board 5, the LED drive board 5 is positioned such that the positions of the sensors 13 correspond to the positions of the LEDs 7, respectively. Here, the sensor drive board 15 and the microplate 21 disposed above the sensor drive board 15 are positioned by a positioning mechanism (not shown).

Incidentally, between the upper surface of the microplate 21 and the LEDs 7, provided is a first aperture substrate 9 having a plurality of openings corresponding to the positions of the wells of the microplate 21. The first aperture substrate 9 is provided to reduce an amount of light emitted from those LEDs 7 (light source) corresponding to the wells other than its associated well (e.g., wells adjacent to the associated well), entering the associated well as external light. The openings of the first aperture substrate 9 are disposed in adjustable positions so that the central axis of each of the openings substantially coincides with the optical axis defined between the associated LED 7 and the associated sensor 13.

On the other hand, between the lower surface of the microplate 21 and the sensors 13, provides is a second aperture substrate 11 having a plurality of openings corresponding to the positions of the wells of the microplate 21. The second aperture substrate 11 is provided to reduce an amount of light (external light) emitted from those LEDs 7 (light source) corresponding to the wells other than its associated well (e.g., wells adjacent to the associated well) and reaching the sensor 13 corresponding to the associated well, when the external light enters the associated well. The openings of the second aperture substrate 11 are disposed in adjustable positions so that the central axis of each of the openings substantially coincides with the optical axis defined between the associated LED 7 and the associated sensor 13.

Because the microplate 21 is placed between the respective sensors 13 on the sensor drive board 15 and the respective LEDs 7 on the LED drive board 5, a microplate reader 27 is configured inside the cell culture space 23 in the housing 3.

The sensor drive board 15 and the LED drive board 5 are powered and controlled by the power supply-control-and-communication unit 19 of FIG. 1. Furthermore, signals of the sensing data detected by the respective sensors 13 are transmitted to a remote or external tablet, smartphone, PC (personal computer), or the like by the power supply-control-and-communication unit 19 (an example of the “signal transmitting unit” in the claims).

Optical measurement and culture environment control by the incubator device 1 is performed, for example, by the following procedure. First, an operator obtains the medium 24 stained with the phenol red, or stains the medium with a desired reagent. Then, the operator places the medium 24 between the LEDs 7 and the sensors 13 (the “sealing step” in the claims) in the housing 3. Subsequently, the LEDs 7 emit light to the medium 24 while maintaining the sealed space of the housing 3, and the light intensity is measured by the sensors 13 as the sensors 13 receive the light from the medium 24 (an example of the “light intensity measuring step” in the claims). The data of the light intensity is transmitted to the remote PC or the like by the power supply-control-and-communication unit 19. The PC or the like (an example of the “absorbance calculation unit”, an example of the “pH calculation unit”, and an example of the “turbidity calculation unit” in the claims) which has received the data of light intensity calculates absorbance and turbidity from the light intensity, and also calculates pH from the absorbance (an example of the “pH calculation step” in the claims). These optical measurements are continuously performed on the medium, and the operator determines the state (condition) of the medium based on the calculated result of pH, turbidity, and the like, and performs treatment such as passage and medium replacement.

As described above, the operator can quantitatively determine the state of the medium, and it becomes possible to perform treatments such as passage and medium replacement at an appropriate timing. Since this is a quantitative determination rather than the operator's experience or visual judgment, it is possible to perform the loading and unloading of the medium container to and from the inside of the housing of the incubator device with a minimum number of times. This reduces or prevents impurity contamination in the medium, and significantly reduces the total time of the operation. Further, since it is not necessary to perform collection of a culture solution as in the prior art, a mechanism for taking out a culture solution becomes unnecessary. Thus, the automation of cell culture can be realized in the future as the management of appropriate culture medium in real time becomes possible.

### Second Embodiment

FIG. 2 shows a second embodiment of an incubator device 31 according to the present invention. When compared to the incubator device 1 of the first embodiment, the incubator device 31 of the second embodiment does not include the first aperture substrate 9 and the second aperture substrate 11, but includes a light guide member 33 to be described below (an example of the “light guide member” in the claims) provided between the lower surface of the microplate 21 and the sensors 13. That is, a microplate reader 39 includes the LED drive board 5, the LEDs 7, the sensors 13, the sensor drive board 15 and the light guide member 33.

The light guide member 33 includes a light guide portion 35 made of a transparent light-transmissive silicone resin (an example of the “light guide path” in the claims), and a light shielding member 37 surrounding the light guide portion (an example of the “light shielding unit” in the claims). The light shielding member 37 is made of a resin of the same material as that of the light guide portion 35, and includes a pigment which is dispersed therein. This pigment (e.g., carbon black) absorbs light.

The inventors have proposed a compact optical measuring device using optical analysis techniques such as absorption spectrophotometry and laser-induced fluorescence method (Patent Literature Document 2). The light guide member 33 employs the structure of the optical unit used in the optical measuring device. As the material of the transparent resin is the same as the material of the pigment-containing resin, the following advantages are obtained; the reflection and scattering at the interface of the two resins is suppressed, and stray light incident on the pigment-containing resin is absorbed by the pigment-containing resin and hardly returns to the light guide path so that complicated multiple reflection of the stray light hardly takes place. The technique of the optical system constructed with silicone resin as described above will be referred to as SOT (Silicone Optical Technologies).

By using the light guide member 33 that employs the SOT structure, and appropriately setting the area of the light inlet (incident end) of the light guide portion 35 and the distance from the light inlet to the light exit of the light guide portion 35, for example, as taught in Patent Literature Document 3, it is possible to suppress the influence of noise light such as undesirable external light incident on the light inlet of the light guide portion 35, and carry out the optical measurement with a ratio of the detected light to the noise light being sufficiently high.

The light guide member 33 in the incubator device 31 shown in FIG. 2 employs the above-described SOT structure, and the light guide portion 35 of the light guide member 33 only transmits light that travels straight along the optical axis of the light guide portion 35. Since oblique incident light is absorbed by the light shielding member 37, the oblique incident light does not pass through the light guide portion 35. Therefore, when the optical axis defined between the sensor unit and the light source unit (LED 7), which correspond to a particular one of the wells (the associated well), substantially coincides with the optical axis of the light guide portion 35, light from those LEDs 7 corresponding to the wells other than the above-mentioned particular one well (e.g., wells adjacent to the above-mentioned particular one well or the associated well) does not enter the sensor 13 in question. This is because the light from the LEDs 7 corresponding to the wells other than the associated well is the light that does not proceed along the optical axis.

According to the experiments conducted by the inventors, the influence of the external light on the measurement results did not change (the influence was negligible) even when the first aperture substrate 9 was removed from the incubator device 1 of the first embodiment. Further, the inventors compared the configuration in which the opening for inserting the medium storage container 21 in the cell culture space 23 inside the housing 3 of the incubator device 1 is left open with the configuration in which the opening is light-shielded, and confirmed that the influence of the external light on the measurement results was only a change of 0.02%. Therefore, the housing 3 may not be light-shielding, or may have a window in the side surface (lateral wall) of the light-shielding housing 3 for observing cells from the outside. The incubator device 31 of the second embodiment need not have the first aperture substrate, and therefore it is easy to visually recognize the cells if the incubator device of the second embodiment is used. Therefore, it is possible to achieve both visual observation and suppression of noise light.

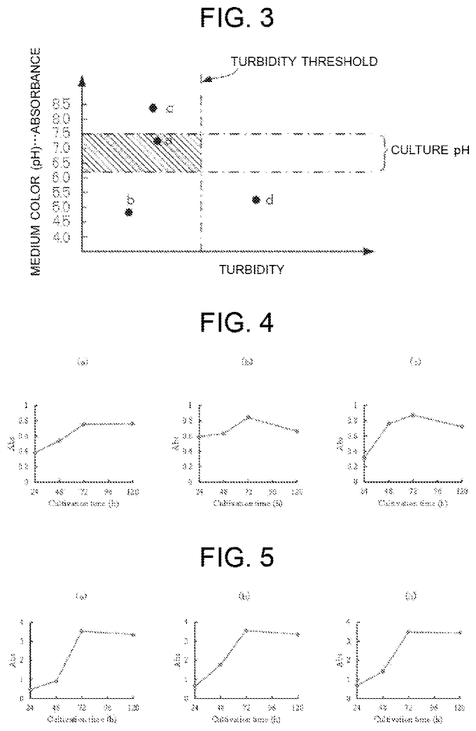

FIG. 3 shows a diagram useful to describe an example of a determination procedure of the measurement result. The vertical axis indicates the pH of the medium and the horizontal axis indicates the turbidity. As an example, we consider a case where cells are seeded in a medium, the medium is stained with the phenol red, and the medium is placed in one of the wells of the microplate. This medium is optically measured, for example, by the incubator device according to the first embodiment or the second embodiment.

Specifically, the optical measurements are conducted to measure the absorbance and the turbidity. First, the color of the medium stained with the phenol red and the pH of the medium are calculated by absorbance measurement (spectrophotometry). If the absorbance measurement indicates that the pH of the medium is greater than, for example, 7.4 (alkaline), it is determined that at least some of the cells in the culture are dead, that the CO₂ concentration in the incubator device is equal to or below a predetermined value, that the circulation of CO₂ in the incubator device is stagnant, and/or that the pH control of the medium is inadequate (Point c in FIG. 3).

In this situation, the operator checks the CO₂ delivery mechanism of the incubator device and the circulation mechanism for circulating CO₂ in the incubator device, and repairs the mechanisms if necessary. If the pH of the medium is alkaline even after the check and repair (maintenance work), it is determined that at least some of the cells in the culture are dead. In this case, a growth factor of the cells in culture may be added to the medium to attempt to revive the cells, but usually, the medium is replaced with a new medium and new cell are seeded.

On the other hand, if the absorbance measurement indicates that the pH of the medium is smaller than, for example, 6.2 (acidic), the result of the turbidity measurement is also considered. When the pH of the medium is determined to be acidic by the absorbance measurement and the turbidity is determined to be higher than an allowable value or a threshold value by turbidity measurement, it is determined that certain impurities enter the medium (impurity contamination in the medium). In this case, since the cell culture is not performed in a desired manner, the operator discards (disposes) the medium from the well in question, in which the cells have been seeded (Point d).

If the pH of the medium is determined to be acidic by the absorbance measurement and the turbidity is determined to be lower than the threshold value by the turbidity measurement, it is determined that the cell culture is performed in the desired manner, and a medium replacement or a passage is performed (Point b).

If it is determined from the absorbance measurement that the pH of the medium is between 6.2 and 7.4, it is determined that the cell culture is proceeding smoothly, the CO₂ condition outside the medium in the incubator device is also good, and there is virtually no impurity contamination in the medium. It is therefore determined that there is no need for medium replacement or passage (Point a).

It should be noted that it is also possible to use the absorbance measurement for the purpose of determining the timing at which the growth factor is introduced into each medium. For example, when the cells to be seeded in the respective wells of the microplate are different from each other, each cell is investigated in advance, and it is possible to determine the above-mentioned timing for each well and to control the medium state of each well.

If the determination of the medium state of the above-described points a to d is performed automatically by a PC or the like (an example of the “carbon dioxide concentration control unit” and an example of the “medium state determination unit” in the claims) rather than the operator, the workload of the operator can be further reduced. The PC or the like is connected to the CO₂ feeding unit of the incubator device via the power supply-control-and-communication unit 19, and adjusts the CO₂ concentration inside the housing 3 on the basis of the calculated PH. Specifically, when the pH is between 6.2 and 7.4, the PC or the like keeps the CO₂ concentration, when the pH is greater than 7.2, the PC or the like increases the CO₂ concentration, and when the pH is smaller than 6.2, the PC or the like reduces the CO₂ concentration (an example of the “carbon dioxide concentration control step” in the claims). A display screen of the PC or the like (an example of the “medium information display unit” in the claims) may display replacement, passage and disposal of the medium which are determined automatically by the PC or the like.

Next, an exemplary configuration of an optical measurement system that can simultaneously measure absorbance and turbidity will be described. White LEDs are used as the light sources. As the sensors, used are RGB color sensors (e.g., Hamamatsu Photonics K. K., Digital Color Sensor: S11059-02DT). When the color sensors manufactured by Hamamatsu Photonics K. K. are used, the sensitivity wavelength range of the Blue channel is between 400 nm and 540 nm, with the maximum sensitivity center wavelength being 460 nm, the sensitivity wavelength range of the Green channel is between 455 nm and 630 nm, with the maximum sensitivity center wavelength being 530 nm, and the sensitivity wavelength range of the Red channel is between 575 nm and 660 nm, with the maximum sensitivity center wavelength being 615 nm.

The turbidity is obtained by measuring the optical density of the 600 nm-wavelength component of the white light, which is directed to each of the wells, with the associated color sensor. The 600 nm-wavelength component is measured using the Green channel or the Red channel. Specifically, the turbidity is calculated by measuring the transmittance change of the 600 nm-wavelength component using the Green channel or the Red channel.

On the other hand, the absorbance is measured using the above-mentioned three channels. The color of the medium is determined based on the absorbance measurement results (transmittance change) of the three channels. When the medium is stained with the phenol red and the pH of the medium changes from the 6.2-7.4 range to 7.4 or more, the color of the medium changes from red to purplish red. When the pH of the medium changes from the 6.2-7.4 range to 6.2 or less, the color of the medium changes from red to yellow. Therefore, the pH of the medium is obtained based on the color of the medium determined from the absorbance measurement.

As described above, since a plurality of calculation processes are carried out simultaneously using the white LEDs as the light sources and the RGB color sensors as the sensors, it becomes possible to simultaneously measure the turbidity and the absorbance (corresponding to the pH of the medium).

By using the incubator device according to one of the embodiments of the present invention, it is also possible to continuously monitor a parameter corresponding to the number of metabolically active cells among the cells seeded in each of the wells of the microplate. Now, an experiment for monitoring a parameter corresponding to the number of metabolically active cells will be described.

Osteoblasts derived from mice were seeded at a seeding density of 5×10⁴ cells/ml in the medium charged (loaded) into each of wells of a microplate. The microplate had twenty-four wells. In addition, tetrazolium salt (WST-1) was added to the medium to investigate the dehydrogenase activity of mitochondria in living cells. Specifically, the absorbance of formazan dye produced upon decomposition of the tetrazolium salt by the dehydrogenase of mitochondria was measured, and the active state of mitochondria was determined.

FIG. 4 shows the absorbance measured for three wells out of the twenty-four wells using the incubator device 31 of the second embodiment. The horizontal axis indicates time (h) and the vertical axis indicates the absorbance (Abs). Absorbance measurements were performed at 24 hours, 48 hours, 72 hours, 120 hours and 168 hours. It should be noted that the wavelength used for absorbance measurement is a blue wavelength (wavelength of the blue light).

Absorbance measurements were also performed using a commercially available spectrophotometer (Ultraviolet-visible spectrophotometer GENESYST™ 10S, manufactured by Thermo Scientific Inc.) at intervals similar to those of the above-described measurements. FIG. 5 shows the results. In this set of drawings, the horizontal axis indicates time (h) and the vertical axis indicates the absorbance (Abs). It should be noted that the measurements were performed by collecting the supernatant liquid from the three wells used for measurement using the above-described incubator device according to the present invention, loading the supernatant liquid into a cuvette and setting the cuvette in the above-mentioned spectrophotometer.

The experimental results from the well, from which the results shown in FIG. 4(a) are obtained, are shown in FIG. 5(a). Similarly, the experimental results from the well, from which the results shown in FIG. 4(b) are obtained, are shown in FIG. 5(b), and the experimental results from the well, from which the results shown in FIG. 4(c) are obtained, are shown in FIG. 5(c). It should be noted that the wavelength used for absorbance measurement by the spectrophotometer is 450 nm.

As is apparent from FIGS. 4 and 5, the measurement results of the incubator device according to the present invention and the measurement results when using the spectrophotometer have a relatively good correlation. In addition, the values of the absorbance increase if the measurements at 24 hours, 48 hours, and 72 hours are looked at. This is because the production quantity of formazan dye increased, and because the whole activity of the dehydrogenase of mitochondria increased. If the used cells are such that this increase in the activity can be considered as an increase in the number of viable cells, the increase in the absorbance is considered as an increase in the number of cell proliferation.

In other words, in the case of the above-described measurement and cells, it becomes possible to continuously monitor an increase in the number of cells by the incubator device according to the present invention. Incidentally, in FIGS. 4 and 5, the inventors think that the reason why the increase in the absorbance after 72 hours is in the saturated trend is because the number of cells in the medium reaches the confluent condition.

### REFERENCE NUMERALS AND SYMBOLS

1 Incubator device
3 Housing
5 LED drive board
7 LED
9 First aperture substrate
11 Second aperture substrate
13 Sensor
15 Sensor drive board
17 Support member
19 Power supply-control-and-communication unit
21 Culture medium storage container (microplate)
23 Cell culture space
24 Culture medium
25 Flange portion
27 Microplate reader
31 Incubator device
33 Light guide member
35 Light guide portion
37 Light shielding member
39 Microplate reader

1. An incubator device for controlling a cell culture environment, the incubator device comprising:
a housing having airtightness;
a light source unit for irradiating at least one medium, in which cells are seeded, with light;
a light measuring unit for measuring intensity of the light from the at least one medium; and
a light guide member for guiding the light from the at least one medium to the light measuring unit,
the light source unit, the light measuring unit and the light guide member being placed inside the housing.

2. The incubator device according to claim 1, wherein the light guide member includes:
a light guide path for transmitting light; and
a light shielding unit provided around the light guide path and configured to shield light, and
the light shielding unit includes a silicone resin and light absorbing particles dispersed in the silicone resin.

3. The incubator device according to claim 1 further including a signal transmitting unit for transmitting intensity of the light measured by the light measuring unit toward an external receiver.

4. The incubator device according to claim 1, wherein the light source unit has a plurality of light sources corresponding to a plurality of said mediums arranged in a microplate, respectively.

5. The incubator device according to claim 1, wherein the light source unit has one or more white LED light sources, and the light measuring unit has one or more RGB color sensors.

6. A cell culture environment control system for controlling a cell culture environment, the system comprising:
an incubator device according to claim 1;
an absorbance calculation unit that calculates an absorbance from light intensity measured by the light measuring unit of the incubator device;
a pH calculation unit that calculates a pH from the absorbance calculated by the absorbance calculation unit; and
a carbon dioxide concentration control unit that maintains a carbon dioxide concentration inside the housing when the pH calculated by the pH calculation unit is within a range from a lower limit value to an upper limit value, increases the carbon dioxide concentration inside the housing when the pH calculated by the pH calculation unit is larger than the upper limit value, and decreases the carbon dioxide concentration inside the housing when the pH calculated by the pH calculation unit is smaller than the lower limit value.

7. The cell culture environment control system according to claim 6 further including:
a turbidity calculation unit that calculates turbidity from the light intensity measured by the light measurement unit;
a medium condition determining unit that determines that disposal of the medium is necessary when the pH calculated by the pH calculation unit is smaller than the lower limit value and the turbidity calculated by the turbidity calculation unit is equal to or larger than a threshold value, determines that replacement or passage of the medium is necessary when the pH calculated by the pH calculation unit is smaller than the lower limit value and the turbidity calculated by the turbidity calculation unit is lower than the threshold value, and determines that replacement of the medium is necessary when the pH calculated by the pH calculation unit is continuously larger than the upper limit value for a predetermined period even if the carbon dioxide concentration inside the housing is increased by the carbon dioxide concentration control unit; and
a medium information display unit that displays necessity of the replacement, the passage or the disposal of the medium determined by the medium condition determining unit.

8. A cell culture environment control method for controlling a cell culture environment, the method comprising:
the sealing step of loading a medium, in which cells are seeded and which is stained with a reagent, in a housing, the housing having a sealed space;
the light intensity measuring step of irradiating the medium with light to measure intensity of light from the medium while maintaining the sealed space;
the pH calculation step of calculating a pH of the medium from the intensity of the light measured in the light intensity measuring step; and
the carbon dioxide concentration control step of maintaining a carbon dioxide concentration inside the housing when the pH calculated in the pH calculation step is within a range from a lower limit value to an upper limit value, increasing the concentration of carbon dioxide inside the housing when the pH calculated in the pH calculation step is larger than the upper limit value, and reducing the carbon dioxide concentration inside the housing when the pH calculated in the pH calculation step is smaller than the lower limit value.

9. The incubator device according to claim 2 further including a signal transmitting unit for transmitting intensity of the light measured by the light measuring unit toward an external receiver.

10. The incubator device according to claim 9, wherein the light source unit has a plurality of light sources corresponding to a plurality of said mediums arranged in a microplate, respectively.

11. The incubator device according to claim 10, wherein the light source unit has one or more white LED light sources, and the light measuring unit is an RGB color sensor.
